(19) United States
(12) Patent Application Publication
(10) Pub. No.: US 2012/0084883 A1
(43) Pub. Date: Apr. 5, 2012

(54) High Oil Content Plants

(76) Inventor: Bart Den Boer, Merelbeke (BE)

(21) Appl. No.: 13/376,919

(22) PCT Filed: Jun. 9, 2010
(86) PCT No.: PCT/EP10/03438

§ 371 Date: Dec. 8, 2011

Related U.S. Application Data
(60) Provisional application No. 61/185,728, filed on Jun. 10, 2009.

(30) Foreign Application Priority Data
Jun. 9, 2009 (EP) 09007588.8

(51) Int. Cl. C12N 15/82; A01H 5/00; A01P 21/00; C12P 1/00; A01N 25/00; C12N 5/04; C11B 1/00

(52) U.S. Cl. 800/281; 435/410; 800/298; 554/8; 435/41; 504/116.1; 435/468

(57) ABSTRACT

Methods and means to modulate oil content and/or oil yield in plants, such as oilseed rape plants are provided. Increased oil content and/or oil yield can be achieved by reducing the functional level or activity of PARP1, e.g. through reduction of the expression of the parp1 gene.

### FIELD OF THE INVENTION

The present invention relates to the field of agricultural products, especially crop plants with increased oil content or oil yield. Provided are methods to increase the oil content or oil yield in plants, plant tissues, plant organs, plant parts, or plant cells by reducing the functional poly(ADP-ribose) polymerase (PARP) activity. Such reduction of the functional PARP activity may be achieved through downregulation of parp1 expression, but may also be achieved in other ways such as for instance chemical inhibition. In particular, methods are provided to increase seed oil content or oil yield in plants, particularly in oilseed rape plants.

### BACKGROUND OF THE INVENTION

Plants are a major source of oils for feed, food, and industrial uses, and the need for vegetable oils will increase with reducing worldwide fossil oil stocks. Hence, there is a need for methods to produce plants with increased oil content.

WO 99/11805 provides recombinant DNA molecules and methods to increase plant oil content by down-regulation of ADP-glucose pyrophosphorylase activity.

U.S. Pat. No. 5,925,805 and U.S. Pat. No. 5,962,767 provide methods to increase seed oil content in plants by expression of a plastid-targeted cytosolic acetyl CoA carboxylase from Arabidopsis, and nucleic acids and DNA constructs for such methods.

Jain et al. (2000) described the enhancement of seed oil content and seed weight in Arabidopsis thaliana by overexpression of glycerol-3-phosphate acyltransferase.

Jako et al. (2001) reported that seed-specific overexpression in Arabidopsis thaliana of diacylglycerol acyltransferase enhanced seed oil content and seed weight. Weselake et al. (2006) and Sharma et al. (2008) describe that transformation of Brassica napus with diacylglycerol acyltransferase-I resulted in increased seed oil content and seed weight.

WO 01/34814, U.S. Pat. No. 6,791,008 and EP 1 230 373 provide methods to increase total oil content in plants by expression of an enzyme with acyl-CoA:diacylglycerol acyltransferase activity.

WO 02/066659 provides a method for increasing the oil content of plants by seed-specific expression of an anti-abscisic acid antibody.

U.S. Pat. No. 6,723,895 and EP 1 283 891 provide methods to increase seed oil content in plants by expression of cytosolic acetyl CoA carboxylase.

Vigeolas et al. (2007) reported the increase in seed oil content and embryo weight in Brassica napus by seed-specific overexpression of glycerol-3-phosphate dehydrogenase from yeast.

WO 03/095655 and WO 2007/051642 provide methods to increase total oil content in a plant by expression of a glycerol-3-phosphate dehydrogenase from yeast.

WO 2004/007727 and U.S. Pat. No. 7,465,850 provide methods to increase total oil content in a plant by expression of a gene encoding a triacylglycerol synthesis-enhancing protein from yeast.

U.S. Pat. No. 7,268,276 and WO 2004/046336 provide methods for increasing the oil content in plants by disruption of the phenylpropanoid pathway.

WO 2004/039946 provides methods for increasing total seed oil level by down-regulation of FAD2 expression.

WO 2004/054351 provides methods for altering oil content in plants by altered expression of the Arabidopsis thaliana At3g52260 gene or an ortholog thereof.

WO 2004/056848, U.S. Pat. No. 7,273,966 and U.S. Pat. No. 7,405,344 provide Brassica sp. plants with increased seed oil levels by expression of a multifunctional fatty acid synthase and a phosphopantetheine protein transferase, and vectors and methods to produce such Brassica plants.

WO 2004/092367 and EP 1 618 193 provide methods to increase total oil content in a plant by expression of a glycerol-3-phosphate acyltransferase activity.

U.S. Pat. No. 7,179,957 provides methods to increase seed oil content in plants by expression of agl11.

U.S. Pat. No. 7,495,150 provides methods to increase seed oil content in plants by down-regulation of homeodomain glabra2 expression.

U.S. Pat. No. 7,179,956 and WO 2005/003312 provide methods to increase corn kernel oil content by expression of a granule bound starch synthase variant.

WO 2008/134402 provides an oilseed rape hybrid line with increased seed oil content.

Still, there remains a need to further increase the oil content of oil seed crops like oilseed rape or provide alternative measures to achieve this goal.

Plants with reduced PARP activity or level are known in the art, and are described for example in WO 00/04173 and WO 2006/133827. However, none of these documents disclose the use of PARP1 expression down-regulation to obtain increased oil content or oil yield in plants.

This problem is solved as herein after described in the different embodiments, examples and claims.

### SUMMARY OF THE INVENTION

The present invention relates to plants with increased total oil content or oil yield. Provided are methods to produce plants with increased total oil content or oil yield by reduction of functional PARP1 activity. Further provided are the use of a PARP1-inhibitory RNA molecule to obtain higher oil content or oil yield in plants, plant tissues, plant organs, plant parts, or plant cells, and the use of plants, plant tissues, plant organs, plant parts, or plant cells with reduced functional PARP1 activity to increase oil yield.

In a first aspect of the invention reduction of functional PARP1 activity may be achieved through down regulation of parp1 gene expression. In one embodiment of the invention, a method is provided to increase total oil content or oil yield of plants, plant tissues, plant organs, plant parts, or plant cells by an introduction of an RNA molecule being capable of down-regulating parp1 gene expression, e.g. through introduction of a chimeric nucleic acid construct comprising a nucleotide region which upon expression yields such RNA molecule. In one embodiment, parpl gene expression is down-regulated by introducing an RNA molecule comprising part of a parp1 encoding nucleotide sequence or a homologous sequence or a chimeric DNA encoding such RNA molecule. In another embodiment, parpl gene expression is down-regulated by introducing an antisense RNA molecule comprising a nucleotide sequence complementary to at least part of a parpl encoding nucleotide or homologous sequence, or by introducing a chimeric DNA encoding such RNA molecule. In yet another embodiment, parp1 gene expression is down-regulated by introducing a double-stranded RNA molecule comprising a sense and an antisense RNA region corresponding to and respectively complementary to at least part of a parp1 gene sequence, which sense and antisense RNA region are capable of forming a double stranded RNA region with each other.

In another embodiment, parp1 gene expression can be down-regulated by introduction of a microRNA molecule (which may be processed from a pre-microRNA molecule) capable of guiding the cleavage of PARP1 mRNA. Again, microRNA molecules may be conveniently introduced into plant cells through expression from a chimeric DNA molecule encoding such miRNA, pre-miRNA or primary miRNA transcript.

In another embodiment of the invention, a method is provided to increase total oil content of plants, plant tissues, plant organs, plant parts, or plant cells by down-regulation of parp1 gene expression through alteration of the nucleotide sequence of the endogenous parp1 gene, such as e.g. alterations in regulatory signals including promoter sequence, intron processing signals, untranslated leader and trailer sequence or polyadenylation signal sequences.

In a second aspect of the invention, down regulation of PARP1 activity may occur at the level of the enzymatic activity. In one embodiment of the invention, a method is provided to increase total oil content of plants, plant tissues, plant organs, plant parts, or plant cells by introduction of a chimeric nucleic acid construct enoding a protein capable of down-regulating PARP1 protein activity. In one embodiment, PARP1 protein activity may be down-regulated by expression of a dominant negative parpl gene. In another embodiment of the invention, PARP1 protein activity may be down-regulated by expression of a PARP1-inactivating antibody.

In another embodiment of the invention, a method is provided to increase total oil content of plants, plant tissues, plant organs, plant parts, or plant cells by down-regulation of PARP1 protein activity through alteration of the nucleotide sequence of the endogenous parp1 gene e.g. through alterations in the coding region introducing, insertions, deletions or substitutions of amino acids or truncations of the encoded protein.

In another embodiment of the invention, a method is provided to increase total oil content of plants, plant tissues, plant organs, plant parts, or plant cells by reducing or inhibiting the PARP1 enzymatic activity through the application of chemical PARP inhibitors.

### DETAILED DESCRIPTION OF THE INVENTION

The present invention is based on the results of field trial investigations demonstrating that plants wherein the functional PARP1 activity is reduced, have an increased total oil content or oil yield.

Accordingly, the current invention provides methods to increase total oil content or oil yield of plants, plant tissues, plant organs, plant parts, or plant cells by reducing the functional PARP1 activity.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region which is functionally or structurally defined may comprise additional DNA regions etc.

In one embodiment of the invention, the plant is an oil crop. Oil crop plants are plants whose oil content is already naturally high and/or which can be used for the production of oils. Non-limiting examples of oil crops are: Arachis hypogaea (peanut), Borago officinalis (borage), Brassica sp., Brassica campestris (mustard), Brassica napus (oilseed rape), Brassica rapa (turnip rape), Camelina sativa (false flax), Cannabis sativa (hemp), Carthamus tinctoris (safflower), Cocos nucifera (coconut), Crambe abyssinica (crambe), Cuphea sp. Elaeis guinensis (African oil palm), Elaeis oleifera (American oil palm), Glycine max (soybean), Gossypium sp. (cotton), Helianthus annuus (sunflower), Jatropha sp., Jatropha curcas (Barbados nut), Juglans sp. (walnut), Linum usitatissimum (flax), Macadamia integrifolia (macadamia), Oenothera biennis (evening primrose), Olea europaea (olive), Oryza sativa (rice), Prunus dulcis (almond), Ricinus communis (castor oil), Sesamum indicum (sesame), Theobroma cacao (cocoa), Triticum sp. (wheat), and Zea mays (corn). In a specific embodiment of the invention, the plant belongs to the Brassica genus. In an even more specific embodiment of the invention, the plant is a Brassica napus plant. The Brassica plant will belong to one of the species Brassica napus, Brassica rapa (or campestris), or Brassica juncea. Alternatively, the plant can belong to a species originating from intercrossing of these Brassica species, such as B. napocampestris, or of an artificial crossing of one of these Brassica species with another species of the Cruciferacea. As used herein “oilseed plant” refers to any one of the species Brassica napus, Brassica rapa (or campestris), or Brassica juncea.

As used herein, “plant part” includes any plant organ or plant tissue, including but not limited to fruits, seeds, embryos, meristematic regions, callus tissue, leaves, roots, shoots, flowers, gametophytes, sporophytes, pollen, and microspores.

In a most preferred embodiment of the invention, the oil content is increased in seeds of a plant. As used herein, “seed” comprises embryo, endosperm and/or seed coat.

As used herein, “total oil” or “oil” of a plant, plant tissue, plant organ, plant part, or plant cell refers to the total of fatty acid, without regard to the type of fatty acid. Total oil comprises triacylglycerides, diacylglycerols, monoacylglycerols, and fatty acids. Thus the “total oil content” or “oil content” of a plant, plant tissue, plant organ, plant part, or plant cell means the content of total oil of that plant, plant tissue, plant organ, plant part, or plant cell and is expressed as fraction of weight of that plant, plant tissue, plant organ, plant part, or plant cell, or alternatively as percentage of weight. The increase in total oil content of a plant, plant tissue, plant organ, plant part, or plant cell by the methods of the present invention is measured relative to the total oil content of a reference plant, plant tissue, plant organ, plant part, or plant cell with similar genetic background. Total oil content can be measured by any appropriate method. Methods to quantify total oil content of plant material are well known in the art and include but are not limited to: near infrared (NIR) analysis, nuclear magnetic resonance (NMR) imaging, Soxhlet extraction, accelerated solvent extraction (ASE), microwave extraction, and supercritical fluid extraction.

“Total oil yield” or “oil yield” as used herein relates to the total oil that is produced by plants, plant tissues, plant organs, plant parts, or plant cells per unit of growing area or growing volume. As an example, seed oil yield expressed in g of oil per m² of growth area is the result of the mathematical product of seed oil content, expressed as fraction of seed weight, weight of individual seeds, expressed in g, seed numbers per plant, and plant number per growing area, expressed in 1/m². As such, an increase in seed oil yield can be the result of a higher mathematical product of seed oil content (unit-less), higher weight of individual seeds (g), higher seed numbers per plant (unit-less), and/or higher plant number per growing area (1/m²).

Total oil content or oil yield can be increased by reducing functional PARP1 activity. For the purpose of the invention, PARP proteins are defined as proteins having poly (ADP-ribose) polymerase activity, i.e. catalyzing the transfer of an ADP-ribose moiety derived from NAD+ mainly to the carboxyl group of an aspartic or glutamic acid residue in the target protein, and subsequent ADP-ribose polymerization. The major target protein is PARP itself, but also histones, high mobility group chromosomal proteins, topoisomerase, endonucleases and DNA polymerases have been shown to be subject to this modification. PARP proteins preferably comprise the so-called “PARP signature”. The PARP signature is an amino acid sequence which is highly conserved between PARP proteins, defined by de Murcia and Ménissier de Murcia (1994) as extending from the amino acid at position 858 to the amino acid at position 906 from the Mus musculus PARP protein. Particularly conserved is the lysine at position 892 of the PARP protein from Mus musculus, which is considered to be involved in the catalytic activity of PARP proteins. Particularly the amino acids at position 865, 866, 893, 898 and 899 of the PARP protein of Mus musculus or the corresponding positions for the other sequences are variable. PARP proteins may further comprise an N-terminal DNA binding domain and/or a nuclear localization signal (NLS).

Currently, two classes of PARP proteins have been described. The first class, as defined herein, comprises the so-called classical Zn-finger containing PARP proteins (ZAP), or PARP1 proteins, encoded by corresponding parpl genes. These proteins range in size from 113-120 kDa and are further characterized by the presence of at least one, preferably two Zn-finger domains located in the N-terminal domain of the protein, particularly located within the about 355 to about 375 first amino acids of the protein. The Zn-fingers are defined as peptide sequences having the sequence CxxCxnHxxC (whereby n may vary from 26 to 30) capable of complexing a Zn atom. Examples of amino acid sequences for PARP proteins from the ZAP class include the sequences which can be found in the PIR protein database with accession number P18493 (Bos taurus), P26466 (Gallus gallus), P35875 (Drosophila melanogaster), P09874 (Homo sapiens), P11103 (Mus musculus), Q08824 (Oncorynchus masou), P27008 (Rattus norvegicus), Q11208 (Sarcophaga peregrina), and P31669 (Xenopus laevis). The nucleotide sequence of the corresponding cDNAs can be found in the EMBL database under accession numbers D90073 (Bos taurus), X52690 (Gallus gallus), D13806 (Drosophila melanogaster), M32721 (Homo sapiens), X14206 (Mus musculus), D13809 (Oncorynchus masou), X65496 (Rattus norvegicus), D16482 (Sarcophaga peregrina), and D14667 (Xenopus laevis). PARP1 proteins have been described in maize (WO 00/04173). In Arabidopsis thaliana, a parp1 gene with AGI number At2g31320 is reported in the TAIR8 protein database.

The second class as defined herein, comprises the so-called non-classical PARP proteins (NAP) or PARP2 proteins, encoded by corresponding parp2 genes. These proteins are smaller (72-73 kDa) and are further characterized by the absence of a Zn-finger domain at the N-terminus of the protein, and by the presence of an N-terminal domain comprising stretches of amino acids having similarity with DNA binding proteins. PARP2 proteins have been reported in maize (WO 00/04173) and in cotton (WO 2006/045633). Two parp2 genes have been identified in the genome of Arabidopsis thaliana (At4g02390 and At5g22470).

The following is a non-limiting list of database entries identifying experimentally demonstrated and putative plant PARP protein sequences that could be identified: AAN12901, AAM13882, CAA10482, AAD20677, BAB09119, CAB80732, CAA88288, AAC19283, Q9ZP54, Q9FK91, Q11207, NP_{—}850165, NP_{—}197639, NP_{—}192148 (Arabidopsis thaliana); CAO70689, CAN75718, CAO48763, CAO40033, A7QVS5, A5AIW8, A7Q0E8, A5AUF8, A7QFD4 (Vitis vinifera); BAF21367, BAC84104, EAZ03601, EAZ39513, BAF08935, EAZ23301, EAY86124, BAD25449, BAD53855, BAD52929, EAZ11816, BAF04898, BAF04897, EAY73948, EAY73947, EAZ11816, EAZ11815, Q7EYV7, Q0E0Q3, A2YKJO, A2X5L4, A2WPQ2, A2WPQ1, A3BIX4, A3A7L2, A2ZSW9, Q5Z8Q9, Q0JMY1, A2ZSW8, NP_{—}001059453, NP_{—}001047021, NP_{—}001042984, NP_{—}001042983 (Oryza sativa); AAC79704, CAA10889, CAA10888, Q9ZSV1, O50017, B4FCJ3 (Zea mays); EDQ65830, EDQ52960, A9SSX0, A9TUE0, A9S9P7 (Physcomitrella patens); AAD51626, Q9SWB4 (Glycine max), Q1SGF1 (Medicago truncatula); ABK93464, A9PAR1 (Populus trichocarpa).

It is clear that other genes or cDNAs encoding PARP1 proteins, or parts thereof, can be isolated from other eukaryotic species or varieties, particularly from other plant species or varieties. Moreover, parp1 genes, encoding PARP1 proteins wherein some of the amino acids have been exchanged for other, chemically similar, amino acids (so-called conservative substitutions), or synthetic parp1 genes (which encode similar proteins as natural parp1 genes but with a different nucleotide sequence, based on the degeneracy of the genetic code) and parts thereof are also suited for the methods of the invention.

As used herein, “functional PARP1 activity” in a plant, plant tissue, plant organ, plant part, or plant cell refers to the PARP1 activity as present in said plant, plant tissue, plant organ, plant part, or plant cell. Functional PARP1 activity is the result of parpl gene expression level and PARP1 enzymatic activity. Accordingly, the functional PARP1 activity in a plant, plant tissue, plant organ, plant part, or plant cell can be reduced by down-regulating parpl gene expression level or by down-regulating PARP1 enzymatic activity, or both and, according to the invention, the increase of oil content or oil yield of a plant, plant tissue, plant organ, plant part, or plant cell can be achieved by down-regulation of parpl gene expression level, by down-regulation of PARP1 enzymatic activity, or both.

Conveniently, parpl gene expression level or PARP1 enzymatical activity is controlled genetically by introduction of chimeric genes altering the parpl gene expression level and/or by introduction of chimeric genes altering the PARP1 enzymatic activity and/or by alteration of the endogenous PARP1-encoding genes.

In accordance with the invention, it is preferred that in order to increase oil content or oil yield, the functional PARP1 activity is reduced significantly, however avoiding that DNA repair (governed directly or indirectly by PARP) is inhibited in such a way that the cells wherein the functional PARP1 activity is reduced cannot recover from DNA damage or cannot maintain their genome integrity. Preferably, the functional PARP activity in the target cells should be decreased about 75%, preferably about 80%, particularly about 90% of the normal level and/or activity in the target cells so that about 25%, preferably about 20%, particularly about 10% of the normal functional PARP activity is retained in the target cells. It is further thought that the decrease in functional PARP activity should not exceed 95%, preferably not exceed 90% of the normal functional PARP activity in the target cells. Methods to determine the content of a specific protein such as the PARP proteins are well known to the person skilled in the art and include, but are not limited to (histochemical) quantification of such proteins using specific antibodies. Methods to quantify PARP activity are also available in the art and include the in vitro assays described by Collinge and Althaus (1994) and Putt and Hergenrother (2004).

Thus in one embodiment of the invention, a method for increasing the oil content or oil yield of a plant, plant tissue, plant organ, plant part, or plant cell comprises the step of down-regulating parpl gene expression. In another embodiment of the invention, a method for increasing the oil content or oil yield of a plant, plant tissue, plant organ, plant part, or plant cell comprises down-regulating PARP1 enzymatic activity.

The term “gene” means any DNA fragment comprising a DNA region (the “transcribed DNA region”) that is transcribed into a RNA molecule (e.g., an mRNA or a pre-miRNA) in a cell under control of suitable regulatory regions, e.g., a plant-expressible promoter. A gene may thus comprise several operably linked DNA fragments such as a promoter, a 5′ leader sequence, a coding region, and a 3′ region comprising a polyadenylation site. An endogenous plant gene is a gene which is naturally found in a plant species.

As used herein a “chimeric nucleic acid construct” refers to a nucleic acid construct which is not normally found in a plant species. A chimeric nucleic acid construct can be DNA or RNA. “Chimeric DNA construct” and “chimeric gene” are used interchangeably to denote a gene which is not normally found in a plant species or to refer to any gene in which the promoter or one or more other regulatory regions of the gene are not associated in nature with part or all of the transcribed DNA region.

The term “gene expression” refers to the process wherein a DNA region under control of regulatory regions, particularly the promoter, is transcribed into an RNA molecule. An RNA molecule is biologically active when it is either capable of interaction with another nucleic acid or protein or which is capable of being translated into a biologically active polypeptide or protein. A gene is said to encode an RNA when the end product of the expression of the gene is biologically active RNA, such as e.g. an antisense RNA, a ribozyme, or a miRNA. A gene is said to encode a protein when the end product of the expression of the gene is a protein or polypeptide. A gene is said to encode a PARP1-inhibitory RNA when the end product of the expression of the gene is capable of down-regulating PARP1 functional activity, i.e. capable of down-regulating parpl gene expression and/or PARP1 protein activity.

For the purpose of the invention, the term “plant-operative promoter” and “plant-expressible promoter” means a promoter which is capable of driving transcription in a plant, plant tissue, plant organ, plant part, or plant cell. This includes any promoter of plant origin, but also any promoter of non-plant origin which is capable of directing transcription in a plant cell.

Promoters that may be used in this respect are constitutive promoters, such as the promoter of the cauliflower mosaic virus (CaMV) 35S transcript (Hapster et al., 1988, Mol. Gen. Genet. 212: 182-190), the CaMV 19S promoter (U.S. Pat. No. 5,352,605; WO 84/02913; Benfey et al., 1989, EMBO J. 8:2195-2202), the subterranean clover virus promoter No 4 or No 7 (WO 96/06932), the Rubisco small subunit promoter (U.S. Pat. No. 4,962,028), the ubiquitin promoter (Holtorf et al., 1995, Plant Mol. Biol. 29:637-649), T-DNA gene promoters such as the octopine synthase (OCS) and nopaline synthase (NOS) promoters from Agrobacterium, and further promoters of genes whose constitutive expression in plants is known to the person skilled in the art.

Further promoters that may be used in this respect are tissue-specific or organ-specific promoters, preferably seed-specific promoters, such as the 2S albumin promoter (Joseffson et al., 1987, J. Biol. Chem. 262:12196-12201), the phaseolin promoter (U.S. Pat. No. 5,504,200; Bustos et al., 1989, Plant Cell 1.(9):839-53), the legumine promoter (Shirsat et al., 1989, Mol. Gen. Genet. 215(2):326-331), the “unknown seed protein” (USP) promoter (Baumlein et al., 1991, Mol. Gen. Genet. 225(3):459-67), the napin promoter (U.S. Pat. No. 5,608,152; Stalberg et al., 1996, Planta 199:515-519), the Arabidopsis oleosin promoter (WO 98/45461), the Brassica Bce4 promoter (WO 91/13980), and further promoters of genes whose seed-specific expression in plants is known to the person skilled in the art.

Other promoters that can be used are tissue-specific or organ-specific promoters like organ primordia-specific promoters (An et al., 1996, Plant Cell 8: 15-30), stem-specific promoters (Keller et al., 1988, EMBO J. 7(12): 3625-3633), leaf-specific promoters (Hudspeth et al., 1989, Plant Mol. Biol. 12: 579-589), mesophyl-specific promoters (such as the light-inducible Rubisco promoters), root-specific promoters (Keller et al., 1989, Genes Dev. 3: 1639-1646), tuber-specific promoters (Keil et al., 1989, EMBO J. 8(5): 1323-1330), vascular tissue-specific promoters (Peleman et al., 1989, Gene 84: 359-369), stamen-selective promoters (WO 89/10396, WO 92/13956), dehiscence zone-specific promoters (WO 97/13865), and the like.

Chimeric RNA constructs according to the invention may be delivered to plant cells using means and methods such as described in WO 90/12107, WO 03/052108 or WO 2005/098004.

In an embodiment of the invention, parpl gene expression is down-regulated by introducing a chimeric DNA construct in the plant, plant tissue, plant organ, plant part, or plant cell, comprising the following operably linked DNA regions:
a) a plant-expressible promoter which functions in a plant, plant tissue, plant organ, plant part, or plant cell;
b) a DNA region which when transcribed yields an PARP1-inhibitory RNA molecule capable of down-regulating parpl gene expression; and
c) a DNA region involved in transcription termination and polyadenylation.

The transcribed DNA region encodes a biologically active RNA which decreases the levels of PARP1 mRNAs available for translation. This can be achieved through well established techniques including co-suppression (sense RNA suppression), antisense RNA, double-stranded RNA (dsRNA), or microRNA (miRNA).

For the purpose of this invention, the “sequence identity” of two related nucleotide or amino acid sequences, expressed as a percentage, refers to the number of positions in the two optimally aligned sequences which have identical residues (x100) divided by the number of positions compared. A gap, i.e. a position in an alignment where a residue is present in one sequence but not in the other, is regarded as a position with non-identical residues. The alignment of the two sequences is performed by the Needleman and Wunsch algorithm (Needleman and Wunsch 1970). The computer-assisted sequence alignment above, can be conveniently performed using standard software program such as GAP which is part of the Wisconsin Package Version 10.1 (Genetics Computer Group, Madison, Wis., USA) using the default scoring matrix with a gap creation penalty of 50 and a gap extension penalty of 3.

It will be clear that whenever nucleotide sequences of RNA molecules are defined by reference to nucleotide sequence of corresponding DNA molecules, the thymine (T) in the nucleotide sequence should be replaced by uracil (U). Whether reference is made to RNA or DNA molecules will be clear from the context of the application.

In one embodiment, parpl gene expression may be down-regulated by introducing a chimeric DNA construct which yields a sense RNA molecule capable of down-regulating parp1 gene expression by co-suppression. The transcribed DNA region will yield upon transcription a so-called sense RNA molecule capable of reducing the expression of a parp1 gene in the target plant or plant cell in a transcriptional or post-transcriptional manner. The transcribed DNA region (and resulting RNA molecule) comprises at least 20 consecutive nucleotides having at least 95% sequence identity to the nucleotide sequence of a PARP1-encoding gene present in the plant cell or plant.

In another embodiment, parpl gene expression may be down-regulated by introducing a chimeric DNA construct which yields an antisense RNA molecule capable of down-regulating parp1 gene expression. The transcribed DNA region will yield upon transcription a so-called antisense RNA molecule capable of reducing the expression of a parpl gene in the target plant or plant cell in a transcriptional or post-transcriptional manner. The transcribed DNA region (and resulting RNA molecule) comprises at least 20 consecutive nucleotides having at least 95% sequence identity to the complement of the nucleotide sequence of a PARP1-encoding gene present in the plant cell or plant.

However, the minimum nucleotide sequence of the antisense or sense RNA region of about 20 nt of the PARP1-encoding region may be comprised within a larger RNA molecule, varying in size from 20 nt to a length equal to the size of the target gene. The mentioned antisense or sense nucleotide regions may thus be about from about 21 nt to about 5000 nt long, such as 21 nt, 40 nt, 50 nt, 100 nt, 200 nt, 300 nt, 500 nt, 1000 nt, 2000 nt or even about 5000 nt or larger in length. Moreover, it is not required for the purpose of the invention that the nucleotide sequence of the used inhibitory PARP1 RNA molecule or the encoding region of the transgene, is completely identical or complementary to the endogenous parpl gene the expression of which is targeted to be reduced in the plant cell. The longer the sequence, the less stringent the requirement for the overall sequence identity is. Thus, the sense or antisense regions may have an overall sequence identity of about 40% or 50% or 60% or 70% or 80% or 90% or 100% to the nucleotide sequence of the endogenous parp1 gene or the complement thereof. However, as mentioned, antisense or sense regions should comprise a nucleotide sequence of 20 consecutive nucleotides having about 95 to about 100% sequence identity to the nucleotide sequence of the endogenous parp1 gene. The stretch of about 95 to about 100% sequence identity may be about 50, 75 or 100 nt.

The efficiency of the above mentioned chimeric genes for antisense RNA or sense RNA-mediated gene expression level down-regulation may be further enhanced by inclusion of DNA elements which result in the expression of aberrant, non-polyadenylated parp1 inhibitory RNA molecules. One such DNA element suitable for that purpose is a DNA region encoding a self-splicing ribozyme, as described in WO 00/01133. The efficiency may also be enhanced by providing the generated RNA molecules with nuclear localization or retention signals as described in WO 03/076619.

In yet another embodiment, parp1 gene expression may be down-regulated by introducing a chimeric DNA construct which yields a double-stranded RNA molecule capable of down-regulating parp1 gene expression. Upon transcription of the DNA region the RNA is able to form dsRNA molecule through conventional base paring between a sense and antisense region, whereby the sense and antisense region are nucleotide sequences as hereinbefore described. dsRNA-encoding parpl expression-reducing chimeric genes according to the invention may further comprise an intron, such as a heterologous intron, located e.g. in the spacer sequence between the sense and antisense RNA regions in accordance with the disclosure of WO 99/53050 (incorporated herein by reference). To achieve the construction of such a transgene, use can be made of the vectors described in WO 02/059294 A1.

In still another embodiment, parpl gene expression is down-regulated by introducing a chimeric DNA construct which yields a pre-miRNA RNA molecule which is processed into a miRNA capable of guiding the cleavage of PARP1 mRNA. miRNAs are small endogenous RNAs that regulate gene expression in plants, but also in other eukaryotes. In plants, these about 21 nucleotide long RNAs are processed from the stem-loop regions of long endogenous pre-miRNAs by the cleavage activity of DICERLIKE1 (DCL1). Plant miRNAs are highly complementary to conserved target mRNAs, and guide the cleavage of their targets. miRNAs appear to be key components in regulating the gene expression of complex networks of pathways involved inter alia in development.

As used herein, a “miRNA” is an RNA molecule of about 20 to 22 nucleotides in length which can be loaded into a RISC complex and direct the cleavage of a target RNA molecule, wherein the target RNA molecule comprises a nucleotide sequence essentially complementary to the nucleotide sequence of the miRNA molecule whereby one or more of the following mismatches may occur:
A mismatch between the nucleotide at the 5′ end of said miRNA and the corresponding nucleotide sequence in the target RNA molecule;
A mismatch between any one of the nucleotides in position 1 to position 9 of said miRNA and the corresponding nucleotide sequence in the target RNA molecule;
Three mismatches between any one of the nucleotides in position 12 to position 21 of said miRNA and the corresponding nucleotide sequence in the target RNA molecule provided that there are no more than two consecutive mismatches. No mismatch is allowed at positions 10 and 11 of the miRNA (all miRNA positions are indicated starting from the 5′ end of the miRNA molecule).

As used herein, a “pre-miRNA” molecule is an RNA molecule of about 100 to about 200 nucleotides, preferably about 100 to about 130 nucleotides which can adopt a secondary structure comprising a dsRNA stem and a single stranded RNA loop and further comprising the nucleotide sequence of the miRNA and its complement sequence of the miRNA* in the double-stranded RNA stem. Preferably, the miRNA and its complement are located about 10 to about 20 nucleotides from the free ends of the miRNA dsRNA stem. The length and sequence of the single stranded loop region are not critical and may vary considerably, e.g. between 30 and 50 nt in length. Preferably, the difference in free energy between unpaired and paired RNA structure is between −20 and −60 kcal/mole, particularly around −40 kcal/mole. The complementarity between the miRNA and the miRNA* do not need to be perfect and about 1 to 3 bulges of unpaired nucleotides can be tolerated. The secondary structure adopted by an RNA molecule can be predicted by computer algorithms conventional in the art such as mFold, UNAFoId and RNAFoId. The particular strand of the dsRNA stem from the pre-miRNA which is released by DCL activity and loaded onto the RISC complex is determined by the degree of complementarity at the 5′ end, whereby the strand which at its 5′ end is the least involved in hydrogen bounding between the nucleotides of the different strands of the cleaved dsRNA stem is loaded onto the RISC complex and will determine the sequence specificity of the target RNA molecule degradation. However, if empirically the miRNA molecule from a particular synthetic pre-miRNA molecule is not functional because the “wrong” strand is loaded on the RISC complex, it will be immediately evident that this problem can be solved by exchanging the position of the miRNA molecule and its complement on the respective strands of the dsRNA stem of the pre-miRNA molecule. As is known in the art, binding between A and U involving two hydrogen bounds, or G and U involving two hydrogen bounds is less strong that between G and C involving three hydrogen bounds.

miRNA molecules may be comprised within their naturally occurring pre-miRNA molecules but they can also be introduced into existing pre-miRNA molecule scaffolds by exchanging the nucleotide sequence of the miRNA molecule normally processed from such existing pre-miRNA molecule for the nucleotide sequence of another miRNA of interest. The scaffold of the pre-miRNA can also be completely synthetic. Likewise, synthetic miRNA molecules may be comprised within, and processed from, existing pre-miRNA molecule scaffolds or synthetic pre-miRNA scaffolds.

WO 2007/131699 (incorporated herein by reference) describes miRNAs to target parp genes and precursors thereof, and methods to generate and use miRNAs to down-regulate parp gene expression.

In another embodiment of the invention, PARP1 protein activity may be down-regulated by introducing a chimeric DNA construct in the plant, plant tissue, plant organ, plant part, or plant cell, comprising the following operably linked DNA regions:
a) a promoter, operative in the plant, plant tissue, plant organ, plant part, or plant cell;
b) a DNA region which when transcribed yields a PARP1-inhibitory RNA molecule capable of down-regulating PARP1 enzymatical activity; and
c) a DNA region involved in transcription termination and polyadenylation.

The transcribed DNA region yields an RNA molecule which may be translated into a biologically active protein capable of decreasing the levels of PARP1 enzymatic activity. This can be achieved e.g. by dominant negative PARP1 mutants or inactivating antibodies to PARP1 proteins.

Thus in one embodiment the PARP1-inhibitory RNA molecule capable of down-regulating endogenous PARP1 protein activity is an RNA molecule which can be translated into dominant negative PARP mutants. “Dominant negative PARP mutants” as used herein, are proteins or peptides comprising at least part of a PARP protein (or a variant thereof), preferably a PARP protein endogenous to the eukaryotic target host cell, which have no PARP enzymatic activity, and which have an inhibitory effect on the activity of the endogenous PARP proteins when expressed in that host cell. Preferred dominant negative PARP mutants are proteins comprising or consisting of a functional DNA binding domain (or a variant thereof) without a catalytic domain, such as the N-terminal Zn finger-containing domain of about 355 to about 375 amino acids of a PARP1 protein. Preferably, dominant negative PARP mutants should retain their DNA binding activity. Dominant negative PARP mutants can be fused to a carrier protein, such as a β-glucuronidase.

In another embodiment the PARP1-inhibitory RNA molecule capable of down-regulating endogenous PARP1 protein activity is an RNA molecule which can be translated into inactivating antibodies to PARP proteins. “Inactivating antibodies to PARP proteins” are antibodies or parts thereof which specifically bind at least to some epitopes of PARP proteins, such as the epitope covering part of the Zn finger II, and which inhibit the activity of the target protein.

It will be clear to the skilled artisan that increase of oil content and/or yield in a plant may also be achieved by inhibiting the enzymatic activitity of PARP1 through chemical means. Chemical inhibitors of PARP are well known and include: 1(2H)-phthalazinone; 1,2-benzopyrone; 1,3-benzodiazine; 1,3-dihydroxynaphthalene; 1,4-Benzoquinone; 1,4-naphthalenedione; 1,5-dihydroxyisoquinoline; 1,8-naphthalimide; 1-hydroxy-2-methyl-4-aminonaphthalene; 1-hydroxyisoquinoline; 1-Indanone; 1-methylnicotinamide chloride; 2,3-benzodiazine; 2,3-dichloro-1,4-naphthoquinone; 2,3-dihydro-1,4-phthalazinedione; 2,3-dihydro-5-hydroxy-1,4-phthalazinedione; 2,4(1 H,3H)-quinazolinedione; 2,6-difluorobenzamide; 2-acetamidobenzamide; 2-amino-3-chloro-1,4-naphthoquinone; 2-Aminobenzamide; 2-bromobenzamide; 2-chlorobenzamide; 2-fluorobenzamide; 2-Hydroxy-1,4-naphthoquinone; 2-hydroxybenzamide; 2-mercapto-4(3H)-quinazolinone; 2-Methoxybenzamide; 2-methyl-1,4-benzopyrone; 2-methyl-1,4-naphthoquinone; 2-Methyl-3-phytyl-1,4-naphthoquinone; 2-methyl-4(3H)-quinazolinone; 2-methylbenzamide; 2-methylchromone; 2-nitro-6(5H)-phenanthridione; 2-phenylchromone; 2-trichloromethyl-4(3H)-quinazolinone; 2H-benz[c]isoquinolin-1-one; 2H-benz[de]isoquinoline-1,3-dione; 3,4-dihydro-1(2H)-naphthalenone; 3,5-dibromosalicylamide; 3,5-dimethoxybenzamide; 3,5-dinitrobenzamide; 3-(N,N-dimethylamino)benzamide; 3-Acetamidobenzamide; 3-acetamidosalicylamide; 3-amino-1,4-dimethyl-5H-pyrido[4,3-b]indole; 3-amino-1-methyl-5H-pyrido[4,3-b]indole; 3-Aminobenzamide; 3-Aminophthalhydrazide; 3-bromobenzamide; 3-Chlorobenzamide; 3-Fluorobenzamide; 3-Guanidinobenzamide; 3-Hydroxybenzamide; 3-Isobutyl-1-methylxanthine; 3-Methoxybenzamide; 3-Methylbenzamide; 3-nitrobenzamide; 3-nitrophthalhydrazide; 3-nitrosalicylamide; 4,8-dihydroxy-2-quinolinecarboxylic acid; 4-amino-1,8-naphthalimide; 4-Aminobenzamide; 4-aminophthalhydrazide; 4-bromobenzamide; 4-chlorobenzamide; 4-chromanone; 4-fluorobenzamide; 4-hydroxy-2-methylquinoline; 4-hydroxy-2-quinolinecarboxylic acid; 4-hydroxybenzamide; 4-Hydroxycoumarin; 4-hydroxypyridine; 4-Hydroxyquinazoline; 4-hydroxyquinoline; 4-methoxybenzamide; 4-methylbenzamide; 4-nitrophthalhydrazide; 5-acetamidosalicylamide; 5-aminosalicylamide; 5-Bromodeoxyuridine; 5-Bromouracil; 5-Bromouridine; 5-chlorosalicylamide; 5-Chlorouracil; 5-Hydroxy-1,4-naphthoquinone; 5-Hydroxy-2-methyl-1,4-naphthoquinone; 5-lodouracil; 5-iodouridine; 5-Methylnicotinamide; 5-Methyluracil; 5-Nitrouracil; 6(5H)-phenanthridinone; 6-aminocoumarin; 6-Aminonicotinamide; 8-acetamidocarsalam; 8-Methylnicotinamide; Acetophenone; alpha-picolinamide; Benzamide; benzoyleneurea; carbonylsalicylamide; carsalam; Chlorthenoxazin; chromone-2-carboxylic acid; cyclohexanecarboxamide; Isonicotinamide; Isonicotinate hydrazide; Isoquinoline; m-acetamidoacetophenone; m-aminoacetophenone; m-hydroxyacetophenone; m-phthalamide; menadione sodium bisulfite; N-(2-chloroethyl)1,8-naphthalamide; N-hydroxynaphthalimide sodium salt; Nicotinamide; Phthalamide; Phthalazine; Pyrazinamide; Quinazoline; reserpine; caffeine; Theobromine; Theophylline; Thiobenzamide; Thionicotinamide or trans-decahydro-1-naphthalenone.

The chimeric DNA construct used to reduce the functional PARP1 activity by down-regulation of parp1 gene expression level and/or by down-regulation of PARP1 protein activity can be stably inserted in a conventional manner into the nuclear genome of a single plant cell, and the so-transformed plant cell can be used in a conventional manner to produce a transformed plant with increased total oil content. In this regard, a T-DNA vector, containing the chimeric DNA construct used to reduce the functional PARP1 activity, in Agrobacterium tumefaciens can be used to transform the plant cell, and thereafter, a transformed plant can be regenerated from the transformed plant cell using the procedures described, for example, in EP 0 116 718, EP 0 270 822, WO 84/02913 and published European Patent application EP 0 242 246 and in Gould et al. (1991). The construction of a T-DNA vector for Agrobacterium mediated plant transformation is well known in the art. The T-DNA vector may be either a binary vector as described in EP 0 120 561 and EP 0 120 515 or a co-integrate vector which can integrate into the Agrobacterium Ti-plasmid by homologous recombination, as described in EP 0 116 718. Preferred T-DNA vectors each contain a promoter operably linked to the transcribed DNA region between T-DNA border sequences, or at least located to the left of the right border sequence. Border sequences are described in Gielen et al. (1984). Introduction of the T-DNA vector into Agrobacterium can be carried out using known methods, such as electroporation or triparental mating. Of course, other types of vectors can be used to transform the plant cell, using procedures such as direct gene transfer (as described, for example in EP 0 223 247), pollen mediated transformation (as described, for example in EP 0 270 356 and WO 85/01856), protoplast transformation as, for example, described in U.S. Pat. No. 4,684,611, plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., 1990; Gordon-Kamm et al., 1990) and rice (Shimamoto et al., 1989; Datta et al. 1990) and the method for transforming monocots generally (WO 92/09696). For cotton transformation, especially preferred is the method described in PCT patent publication WO 00/71733. For rice transformation, reference is made to the methods described in WO 92/09696, WO 94/00977 and WO 95/06722. The resulting transformed plant can be used in a conventional plant breeding scheme to produce more transformed plants with increased total oil content.

In another embodiment of the invention, the functional activity of PARP1 may be reduced by modification of the nucleotide sequence of the endogenous parp1 genes. In a preferred embodiment, the PARP1-encoding DNA sequence is altered so that the encoded mutant PARP1 proteins retain about 10% of their activity. In another preferred embodiment, the parp1 gene expression-regulating sequences are altered so that the parp1 gene expression levels are down-regulated.

Methods to achieve such a modification of endogenous parp1 genes include homologous recombination to exchange the endogenous parpl genes for mutant parp1 genes e.g. by the methods described in U.S. Pat. No. 5,527,695. In a preferred embodiment such site-directed modification of the nucleotide sequence of the endogenous parp1 genes is achieved by introduction of chimeric DNA/RNA oligonucleotides as described in WO 96/22364 or U.S. Pat. No. 5,565,350.

Methods to achieve such a modification of endogenous parp1 genes also include mutagenesis. It will be immediately clear to the skilled artisan, that mutant plant cells and plant lines, wherein the functional PARP1 activity is reduced may be used to the same effect as the transgenic plant cells and plant lines described herein. Mutants in parp1 gene of a plant cell or plant may be easily identified using screening methods known in the art, whereby chemical mutagenesis, such as e.g., EMS mutagenesis, is combined with sensitive detection methods (such as e.g., denaturing HPLC). An example of such a technique is the so-called “Targeted Induced Local Lesions in Genomes” method as described in McCallum et al, Plant Physiology 123 439-442 or WO 01/75167. However, other methods to detect mutations in particular genome regions or even alleles, are also available and include screening of libraries of existing or newly generated insertion mutant plant lines, whereby pools of genomic DNA of these mutant plant lines are subjected to PCR amplification using primers specific for the inserted DNA fragment and primers specific for the genomic region or allele, wherein the insertion is expected (see e.g. Maes et al., 1999, Trends in Plant Science, 4, pp 90-96). Thus, methods are available in the art to identify plant cells and plant lines comprising a mutation in the parpl gene. This population of mutant cells or plant lines can then be tested for functional PARP1 activity and oil content and compared to non-mutated cells or plant lines with similar genetic background.

According to a particular embodiment of the invention, the transformed plant cells and plants obtained by the methods of the invention may contain, in addition to the chimeric DNA construct from the invention, at least one other chimeric gene containing a nucleic acid encoding a protein of interest. Examples of such proteins of interest include an enzyme for resistance to a herbicide, such as the bar or pat enzyme for tolerance to glufosinate-based herbicides (EP 0 257 542, WO 87/05629 and EP 0 257 542, White et al. 1990), the EPSPS enzyme for tolerance to glyphosate-based herbicides such as a double-mutant corn EPSPS enzyme (U.S. Pat. No. 6,566,587 and WO 97/04103), or the HPPD enzyme for tolerance to HPPD inhibitor herbicides such as isoxazoles (WO 96/38567).

The transformed plant cells and plants obtained by the methods of the invention may also contain, in addition to the chimeric DNA construct from the invention, at least one other chimeric gene which confers increased oil content, or they may be obtained by methods to increase oil content. Non-limiting examples of such chimeric genes and/or methods are provided in U.S. Pat. No. 5,925,805, U.S. Pat. No. 6,723,895, WO 02/066659, US 2006/0168684, WO 2004/007727, WO 2004/039946, U.S. Pat. No. 7,405,344, WO 2004/092367, U.S. Pat. No. 7,179,957, US 2005/0278805, WO 2005/003312, and WO 2008/134402.

A further embodiment of the present invention relates to the use of a plant, plant tissue, plant organ, plant part, or plant cell with reduced functional PARP1 activity to increase oil yield.

The transformed plant cells and plants obtained by the methods of the invention may be further used in breeding procedures well known in the art, such as crossing, selfing, and backcrossing. Breeding programs may involve crossing to generate an F1 (first filial) generation, followed by several generations of selfing (generating F2, F3, etc.). The breeding program may also involve backcrossing (BC) steps, whereby the offspring is backcrossed to one of the parental lines, termed the recurrent parent.

The transformed plant cells and plants obtained by the methods of the invention may also be further used in subsequent transformation procedures.

The transformed plant cells and plants obtained by the methods of the invention may also be treated with herbicides including Clopyralid, Diclofop, Fluazifop, Glufosinate, Glyphosate, Metazachlor, Trifluralin Ethametsulfuron, Quinmerac, Quizalofop, Clethodim, Tepraloxydim; with fungicides, including Azoxystrobin, Carbendazim, Fludioxonil, Iprodione, Prochloraz, Vinclozolin or with insecticides, including Carbofuran, Organophosphates, Pyrethroids, Thiacloprid, Deltamethrin, Imidacloprid, Clothianidin, Thiamethoxam, Acetamiprid, Dinetofuran, β-Cyfluthrin, gamma and lambda Cyhalothrin, tau-Fluvaleriate, Ethiprole, Spinosad, Spinotoram, Flubendiamide, Rynaxypyr, Cyazypyr or 4-[[(6-Chlorpyridin-3-yl)methyl](2,2-difluorethyl)amino]furan-2(5H)-on.

The following non-limiting examples describe the characteristics of oilseed rape plants obtained in accordance with the invention. Unless otherwise stated, all recombinant DNA techniques are carried out according to standard protocols as described in Sambrook et al. (1989) Molecular Cloning: A Laboratory Manual, Second Edition, Cold Spring Harbour Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy published by BIOS Scientific Publications Ltd (UK) and Blackwell Scientific Publications, UK.

In the description and examples, reference is made to the following sequences:
SEQ ID No.: 1: DNA sequence of the T-DNA vector pTYG48
SEQ ID No.: 2: DNA sequence of the parp-1 cDNA from Arabidopsis thaliana
SEQ ID No.: 3: Protein sequence of PARP-1 from Arabidopsis thaliana

### EXAMPLES

### Example 1

### Construction of the p35S::(dsRNA-AtPARP1) Chimeric Gene and T-DNA Vector Comprising this Gene

Using standard recombinant DNA procedures, the following DNA regions were operably linked:
a CaMV 35S promoter region (Odell et al., 1985)
a Cab22 leader region (Harpster et al., 1988)
an AtPARP1 sense RNA-encoding region (from nt 418 to nt 1459 of the open reading frame; SEQ ID No.: 2)
an AtPARP1 antisense RNA-encoding region (from nt 946 to nt 418 of the open reading frame; SEQ ID No.:2)
a CaMV35S 3′ end region (Mogen et al., 1990)

Using appropriate restriction enzymes, the p35S::(dsRNA-AtPARP1) chimeric gene was introduced in the polylinker between the T-DNA borders of a T-DNA vector derived from pGSV5 (WO 97/13865) together with a chimeric bar marker gene consisting of the following operably-linked DNA regions:
an Act2 promoter region (An et al., 1996)
a phosphinotricin acetyltransferase encoding DNA (U.S. Pat. No. 5,646,024)
a 3′ end region of a nopaline synthase gene (Depicker et al., 1982)

The resulting T-DNA vector was introduced in Agrobacterium tumefaciens C58C1 Rif(pGV4000) by electroporation as described by Walkerpeach and Velten (1995) and transformants were selected using spectinomycin and streptomycin.

### Example 2

### Agrobacterium-mediated Transformation of Brassica napus with the T-DNA Vectors of Example 1

The Agrobacterium strains were used to transform the Brassica napus var. N90-740 applying the hypocotyl transformation method essentially as described by De Block et al. (1989), except for the following modifications:
Hypocotyl explants were precultured for 1 day on A2 medium [1×MS; 0.5 g/l 2-(N-morpholino)ethanesulfonic acid (MES); 1.2% glucose; 0.5% agarose; 1 mg/l 2,4-D; 0.25 mg/l naphthalene acetic acid (NAA); 1 mg/l 6-benzylaminopurine (BAP); pH5.7].
Infection medium was A3 [1×MS; 0.5 g/l MES; 1.2% glucose; 0.1 mg/l NAA; 0.75 mg/l BAP; 0.01 mg/l gibberellinic acid (GA3); pH5.7].
Selection medium was A5G [1×MS; 0.5 g/l MES; 1.2% glucose; 40 mg/l adenine.SO₄; 0.5 g/l polyvinylpyrrolidone (PVP); 0.5% agarose; 0.1 mg/l NAA; 0.75 mg/l BAP; 0.01 mg/l GA3; 250 mg/l carbenicillin; 250 mg/l triacillin; 5 mg/l AgNO₃; pH5.7]. After three weeks, selection is continued on A5J medium [A5G with additional 1.8% sucrose].
Regeneration medium was A6 [MS; 0.5 g/l MES; 2% sucrose; 40 mg/l adenine.SO₄; 0.5 g/l PVP; 0.5% agarose; 0.0025 mg/l BAP; 250 mg/l triacillin; pH5.7].
Healthy shoots were transferred to rooting medium A9 [0.5×MS; 1.5% sucrose; 100 mg/l triacillin; 0.6% agar; pH5.8] in 1 liter vessels.
   MS stands for Murashige and Skoog medium (Murashige and Skoog, 1962)

Trangenic plants were selected for glufosinate resistance and verified using Southern blotting, PCR and RT-PCR. After three generations of self-pollination, glufosinate-resistant plants were crossed with Brassica napus var. “Simon”. After two generations of self-pollination of the resulting hybrids, homozygous and azygous transformants were isolated and self-pollinated. The resulting generation was used in field trials.

### Example 3

### PARP Expression Analysis in Brassica napus Transformants

To evaluate the efficiency of parp-1 gene silencing in transformants, genotoxic stress was induced in leaf segments by incubation with bleomycin for 6 h. Bleomycin induces single and double strand breaks in DNA, and thus can be used to induce parp-1 gene expression (Povirk,1996, Mutation Research 355:71-89).

For this, leaf segments were incubated with gentle shaking for 6 h in the dark in M205 [0.5×MS; 0.5 g/l MES; 0.5×B5 vitamins, 1% sucrose; pH 5.6] containing 1.5, 0.75, 0.25 μg/ml bleomycin, or no bleomycin. RNA was isolated according to standard procedures. First strand cDNA was used as a template for quantitative RT-PCR to monitor parp-1 gene expression. Expression of parp1 was induced considerably upon genotoxic stress in wild-type lines, but induction of parp1 in transgenic lines was several fold lower upon genotoxic stress.

### Example 4

### Oil Content and Composition of Brassica napus Transformants

Two transgenic lines, representing two independent transgenic events were field-trialed at one location during one growing season. The most promising event was field-trialed at two different locations the year thereafter. Results from 3 different plots were averaged and differences were statistically analysed. Figures are indicated in bold when the 1-tailed t-test is statistically significant (P<0.05).

Growing season 1—Field Trial Site 1

| | Oil % | Protein % | Total Gluc. | C18:1 | C18:2 | C18:3 | C22:1 | Sats |
|---|---|---|---|---|---|---|---|---|
| EVENT | Average | Average | Average | Average | Average | Average | Average | Average |
| Event 1- azygotic segregant | 45.27 | 49.57 | 9.45 | 57.80 | 19.67 | 9.87 | 3.00 | 7.43 |
| Event 1 - transgenic line | 47.13 | 51.67 | 12.43 | 58.4 | 19.53 | 10.60 | 2.4 | 6.97 |
| P-value 1-tailed | 0.0028 | 0.0012 | 0.0343 | 0.3174 | 0.382 | 0.0257 | 0.1115 | 0.0089 |
| P-value 2-tailed | 0.0056 | 0.0024 | 0.0687 | 0.6348 | 0.7641 | 0.0514 | 0.223 | 0.0177 |
| Event 2- azygotic segregant | 45.23 | 51.10 | 15.73 | 58.40 | 20.63 | 9.23 | 2.57 | 7.13 |
| Event 2 - transgenic line | 49.93 | 52.53 | 9.68 | 61.13 | 16.47 | 10.63 | 2.33 | 6.77 |
| P-value 1-tailed | 0.0001 | 0.052 | 0.0161 | 0.0052 | 0.0001 | 0.0006 | 0.3829 | 0.0089 |
| P-value 2-tailed | 0.0001 | 0.104 | 0.0321 | 0.0104 | 0.0003 | 0.0012 | 0.7658 | 0.0177 |

Growing season 2—Field Trial Site 2

| | Oil % | Protein % | Total Gluc. | C18:1 | C18:2 | C18:3 | C22:1 | Sats |
|---|---|---|---|---|---|---|---|---|
| EVENT | Average | Average | Average | Average | Average | Average | Average | Average |
| Event 2- azygotic segregant | 43.60 | 46.98 | 9.76 | 59.92 | 21.73 | 7.99 | 2.21 | 7.36 |
| Event 2 - transgenic line | 49.44 | 48.12 | 5.84 | 64.35 | 18.89 | 7.86 | 1.05 | 6.92 |
| P-value 1-tailed | 0.0022 | 0.2625 | 0.0021 | 0.0257 | 0.0038 | 0.36 | 0.038 | 0.0376 |
| P-value 2-tailed | 0.0045 | 0.525 | 0.0042 | 0.0515 | 0.0076 | 0.7276 | 0.076 | 0.572 |

Growing season 3—Field Trial Site 3

| | Oil % | Protein % | Total Gluc. | C18:1 | C18:2 | C18:3 | C22:1 | Sats |
|---|---|---|---|---|---|---|---|---|
| EVENT | Average | Average | Average | Average | Average | Average | Average | Average |
| Event 2- azygotic segregant | 44.01 | 45.88 | 11.83 | 59.63 | 21.25 | 7.99 | 1.91 | 7.25 |
| Event 2 - transgenic line | 50.09 | 46.65 | 5.89 | 65.77 | 18.08 | 7.69 | 0.933 | 6.86 |
| P-value 1-tailed | 0.0004 | 0.2168 | 0.0043 | 0.0061 | 0.0066 | 0.18 | 0.092 | 0.0011 |
| P-value 2-tailed | 0.0009 | 0.4336 | 0.0086 | 0.0123 | 0.0132 | 0.3605 | 0.1841 | 0.0023 |

### Example 5

### Seed and Oil Yield of Brassica nasus Transformants

### Growing Season 1—Field Trial Site 1

| | | Thousand Seed | | | |
|---|---|---|---|---|---|
| | Oil % | Weight (g) | Seed Yield (kg/ha) | Oil Yield (kg/ha) | |
| EVENT | Average | Average | Average | Average | % |
| Event 1-azygotic segregant | 45.27 | 3.88 | 1347.52 | 610.02 | 100 |
| Event 1-transgenic line | 47.13 | 3.90 | 1492.20 | 703.27 | 115.3 |
| P-value 1-tailed | 0.0028 | 0.4476 | 0.0834 | 0.0433 | |
| P-value 2-tailed | 0.0056 | 0.8951 | 0.1668 | 0.0866 | |
| Event 2-azygotic segregant | 45.23 | 4.00 | 1719.51 | 777.56 | 100 |
| Event 2-transgenic line | 49.93 | 3.76 | 2056.56 | 1026.84 | 131.4 |
| P-value 1-tailed | 0.0001 | 0.0736 | 0.0082 | 0.0017 | |
| P-value 2-tailed | 0.0001 | 0.1472 | 0.0164 | 0.0034 | |

### Growing Season 2—Field Trial Site 2

| | | Thousand Seed | | | |
|---|---|---|---|---|---|
| | Oil % | Weight (g) | Seed Yield (kg/ha) | Oil Yield (kg/ha) | |
| EVENT | Average | Average | Average | Average | % |
| Event 2-azygotic segregant | 43.60 | 4.69 | 1252.49 | 546.61 | 100 |
| Event 2-transgenic line | 49.44 | 4.40 | 1391.71 | 692.87 | 126.99 |
| P-value 1-tailed | 0.0022 | 0.1217 | 0.32 | 0.1976 | |
| P-value 2-tailed | 0.0045 | 0.2434 | 0.6565 | 0.3952 | |

### Growing Season 3—Field Trial Site 3

| | | Thousand Seed | | | |
|---|---|---|---|---|---|
| | Oil % | Weight (g) | Seed Yield (kg/ha) | Oil Yield (kg/ha) | |
| EVENT | Average | Average | Average | Average | % |
| Event 2-azygotic segregant | 44.01 | 4.98 | 2158.09 | 949.78 | 100 |
| Event 2-transgenic line | 50.09 | 4.60 | 3118.41 | 1560.32 | 164.28 |
| P-value 1-tailed | 0.0004 | 0.0207 | 0.0023 | 0.0005 | |
| P-value 2-tailed | 0.0009 | 0.0413 | 0.0047 | 0.0011 | |

From the data in Examples 5 and 6, it will be clear that oil content and oil yield consistently and in a statistically significant way are higher in the transgenic lines than in their azygotic counterpart (control) plants.

### Example 6

### Oil Content and Composition in Brassica navus Transformants

Greenhouse grown Brassica seeds from transgenic event 2 and corresponding control plants were analysed by NIR for oil content. Again oil content (although lower than field grown seed) is significantly higher in transgenic samples than in the control samples.

| EVENT | Oil % | Protein % | Total Gluc. |
|---|---|---|---|
| Event 2-azygotic segregant | Average | Average | Average |
| sample 1 | 40.2 | 27.9 | 6.2 |
| sample 2 | 41.3 | 27.9 | 6.3 |
| sample 3 | 40 | 28.2 | 2.5 |
| sample 4 | 41.9 | 27.5 | 7.9 |
| sample 5 | 40.3 | 27.7 | 4.6 |

| EVENT | Oil % | Protein % | Total Gluc. |
|---|---|---|---|
| Event 2-transgenic line | Average | Average | Average |
| sample 1 | 43.3 | 26.6 | 5.8 |
| sample 2 | 43 | 26.2 | 5 |
| sample 3 | 43.7 | 25.8 | 10 |
| sample 4 | 42.8 | 26.49 | 4.9 |
| sample 5 | 42.7 | 26.8 | 7.6 |

### References

Collinge, M. A. and Althaus, F. R. (1994) Expression of human poly(ADP-ribose) polymerase in Saccharomyces cerevisiae. Mol. Gen. Genet. 245:686-693.

Jain, R. K., Coffey, M., Lai, K., Kumar, A., and MacKenzie, S. L. (2000) Enhancement of seed oil content by expression of glycerol-3-phosphate acyltransferase genes. Biochem. Soc. Trans. 28:958-961.

Jako, C., Kumar, A., Wei, Y., Zou, J., Barton, D. L., Giblin, E. M., Covello, P. S., and Taylor, D. C. (2001) Seed-Specific Over-Expression of an Arabidopsis cDNA Encoding a Diacylglycerol Acyltransferase Enhances Seed Oil Content and Seed Weight. Plant Phys. 126:861-874.

Putt, K. S. and Hergenrother, P. J. (2004) An enzymatic assay for poly(ADP-ribose) polymerase-1 (PARP-1) via the chemical quantitation of NAD+: application to the high-throughput screening of small molecules as potential inhibitors. Anal. Biochem. 326:78-86.

Sharma, N., Anderson, M., Kumar, A., Zhang, Y., Giblin, E. M., Abrams, S. R., Zaharia, L. I., Taylor, D. C., Fobert, P. R. (2008) Transgenic increases in seed oil content are associated with the differential expression of novel Brassica-specific transcripts. BMC Genomics 9:619-636.

Vigeolas, H., Waldeck, P., Zank, T., and Geigenberger, P. (2007) Increasing seed oil content in oil-seed rape (Brassica napus L.) by over-expression of a yeast glycerol-3-phosphate dehydrogenase under the control of a seed-specific promoter. Plant Biotech. J. 5:431-441.

Weselake, R. J., Shah, S., Taylor, D. C., Zou, J., Laroche, A., Moloney, M. M., Rakow, G., Raney, P., and Harwood, J. (2006) Transformation of Brassica napus with diacylglycerol acyltransferase-I results in increased seed oil content. In Current Advances in the Biochemistryand Cell Biology of Plant Lipids Edited by: Benning C., Ohlrogge, J. Salt Lake City: Aardvark Global Publishing Co; 232-234.

1. A method for increasing the oil content or oil yield in a plant, plant tissue, plant organ, plant part, or plant cell, said method comprising the step of reducing functional PARP1 activity in cells of said plant, plant tissue, plant organ, plant part or in said cell.

2-7. (canceled)

8. The method according to claim 1, comprising introducing an RNA molecule in said plant, plant tissue, plant organ, plant part, or plant cell, wherein said RNA molecule comprises a PARP1-inhibitory RNA molecule capable of down-regulating the expression of said parp1 gene.

9. The method of claim 1, comprising introducing a chimeric DNA construct in said plant, plant tissue, plant organ, plant part, or plant cell, wherein said chimeric DNA construct comprises the following operably linked DNA regions:
a) a promoter, operative in said plant, plant tissue, plant organ, plant part, or plant cell;
b) a transcribed DNA region, which when transcribed yields a PARP1-inhibitory RNA molecule, said PARP1-inhibitory RNA molecule being capable of down-regulating the expression of said parp1 gene;
c) a DNA region involved in transcription termination and polyadenylation.

10. The method of claim 9, wherein said transcribed DNA region encodes a sense RNA molecule, said DNA region comprising a nucleotide sequence of at least 20 nucleotides with at least 95% identity to the DNA strand of said parp1 gene or comprising a nucleotide sequence of at least 20 nucleotides with at least 95% identity to the complement of the DNA strand of said parp1 gene.

11. (canceled)

12. The method of claim 9, wherein said transcribed DNA region encodes a double-stranded RNA molecule, comprising:
d) a sense RNA region comprising at least 20 consecutive nucleotides having at least 95% identity to said parp1 gene;
e) an antisense RNA region comprising at least 20 nucleotides complementary to said sense RNA region;
wherein said sense and antisense RNA regions are capable of forming a double stranded RNA region and wherein said double-stranded RNA molecule is capable of down-regulating the expression of said parp1 gene.

13. The method of claim 0, wherein said transcribed DNA region encodes a pre-miRNA molecule which is processed into a miRNA capable of guiding the cleavage of mRNA transcribed from said parp1 gene.

14. The method of claim 1, comprising introducing a chimeric DNA construct in said plant, plant tissue, plant organ, plant part, or plant cell, wherein said chimeric DNA construct comprises the following operably linked DNA regions:
f) a promoter, operative in said plant, plant tissue, plant organ, plant part, or plant cell;
g) a transcribed DNA region, which when transcribed yields a PARP1-inhibitory RNA molecule, said PARP1-inhibitory RNA molecule being capable of down-regulating said PARP1 protein activity;
a DNA region involved in transcription termination and polyadenylation.

15. The method of claim 0, wherein said transcribed DNA region encodes for a dominant negative PARP1 mutant capable of reducing PARP1 protein activity or for an inactivating antibody to PARP1 proteins capable of reducing PARP1 protein activity.

16-18. (canceled)

19. The method of claim 1, comprising altering the nucleotide sequence of the endogenous parp1 gene.

20. Use of a PARP1-inhibitory RNA molecule to obtain higher oil content or oil yield in a plant, plant tissue, plant organ, plant part, or plant cell.

21. Use of a nucleotide sequence comprising at least 19 out of 20 consecutive nucleotides of the nucleotide sequence of SEQ ID No. 2 or of a nucleotide sequence encoding part of a protein with the amino acid sequence of any one of SEQ ID No. 3, or of the complement of one of said nucleotide sequences, in a plant, plant tissue, plant organ, plant part, or plant cell to increase oil content or oil yield.

22. (canceled)

23. Use of a plant, plant tissue, plant organ, plant part, or plant cell with reduced functional PARP1 activity to increase oil content or oil yield.

24. A method for producing oil comprising growing or culturing plant, plant tissue, plant organ, plant part, or plant cell with reduced functional PARP1 activity and recovering oil from said plant, plant tissue, plant organ, plant part, or plant cell.

25. The method of claim 1 wherein said reduction of functional PARP 1 activity is achieved by applying a chemical inhibitor of PARP.


SEQUENCE LISTING (3 sequences)

SEQ ID NO: 1 - DNA, 11585 bp, Artificial
  DNA sequence of the T-DNA vector pTYG48
cggcaggatatattcaattgtaaatggctccatgggcaattggtacctacctagaggatcttcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcttcggatcctagacgcgtgagatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggtccatggtttatgagcttggaaagaaagagcggaagaagatgagattgaggaagattcaaaacggagaatgaatctcctggattatctcttactttctctcttagtcttcttccttgttcttctctgtcaagtcgccggagattcaaaacggctgatgaaagtgaggaggacaacgagacaattcaaagcggagaggaaaatatatgaatttatataggcgggtttatctcttacaactttattttcggcctttcaaaaaaataattaaaatcgacagacacgaatcatttcgaccacaggtaaagataacgtgacctggctgtcagacagccttttccctcgtgttaactaatttttaaactaattaatcatctcagcccttggattagttcttttgctttgatggcttcatgactgtgacctgctcgatccgcgtgttacatgacagctccgtttttttagtggttaacttaaaccgagtcaatccaggcaacgttagtcgtcgtcgtggttggcttgttcaattagatttcatacaattcaacgtaatttaattcgttttctattagaattgtatcataattaattcagaccgtgaaagaaagtgtctttcatgatgtgtttatggatatttatacaataagatacaatgtttcatcatattcactattcacgattagtatgtacattaaataatggctactactacatccgaactcgtcaaaacgattctgaatcaattatacatatgctgactcttgcatacataaaaaatagttgtttaaattttgtctaactaatgtttggtataagtataatgttgagttgagataccaattacatcgagtctagccattttgtcgtnncatattcgtcaaaactttcttacataatgataacctagatctagatgagatatgtatcaatgtatttgagatcataatgcggccgtatgcatggcgcgccatatgcaccatacatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactcgagctcatttctctattacttcagccataacaaaagaactcttttctcttcttattaaaccaaaaccttggcggccgctacgtatattctccaagcctgaaggcccgggtaacaaaggtttgatgtggcatcacgctaaatgtttccttgaaatgtcttcctctactgaactggaaagtttgtctggatggagaagtataccagactcagaccaagaagctcttcttcccttagtgaagaaagctctgccggcagccaaaactgagacagcagaagcacgtcaaacaaattcaagagcaggcacaaaacgaaaaaatgattctggtgataacgagaagtcgaaactagcaaaaagtagttttgacatgtctacaagtggtgctttacaaccttgtagcaaagaaaaggaaatggaggcccaaactaaggaattgtgggacctgaaggatgatctgaaaaaatatgtaacatcagctgagttgcgggaaatgcttgaagtaaatgaacaaagtacaagaggatctgaacttgatctgcgtgataaatgtgctgatggcatgatgtttggcccactcgctctctgcccaatgtgctctgggcatctttctttctccggaggactttaccgatgccatggatacatctcagaatggagcaaatgttctcattccactttggatccagaccgcatcaaagggaagtggaaaatccctgacgaaacagaaaatcaattccttctgaagtggaataagtctcaaaagagtgtgaagccaaaacgtattctgcgtcctgtattgtctggcgagacatctcagggtcaaggttctaaagatgcaactgactcctcaaggagtgaaaggctagcagatcttaaagtttcaattgctggaaatactaaggaaaggcaaccatggaagaagagaattgaggaagctggtgcagagtttcatgctaatgttaaaaaaggtgcaagctgtttggttgtttgtggactgacagatatcagagacgctgaaatgagaaaggcaaggaggatgaaagtggcaatcgtgagagaggattatttggttgattgttttaaaaaacagaggaaacttccatttgacaagtacaaaattgaagacactaggtagaattccggagaaagaaagatgcccagagcacattgggcagagagcgagtgggccaaacatcatgccatcagcacatttatcacgcagatcaagttcagatcctcttgtactttgttcatttacttcaagcatttcccgcaactcagctgatgttacatattttttcagatcatccttcaggtcccacaattccttagtttgggcctccatttccttttctttgctacaaggttgtaaagcaccacttgtagacatgtcaaaactactttttgctagtttcgacttctcgttatcaccagaatcattttttcgttttgtgcctgctcttgaatttgtttgacgtgcttctgctgtctcagttttggctgccggcagagctttcttcactaagggaagaagagcttcttggtctgagtctggtatacttctccatccagacaaactttccagttcagtagaggaagacatttcaaggaaacatttagcgtgatgccacatcaaacctttgttacccgggccttcaggcttggagaatatacaattccccgggtctagagctagcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcatgcatggtacagcggccctagagcgactggcaggatatataccgttgtaatttgtcgcgtgtgaataagtcgctgtgtatgtttgtttgattgtttctgttggagtgcagcccatttcaccggacaagtcggctagattgatttagccctgatgaactgccgaggggaagccatcttgagcgcggaatgggaatggatttcgttgtacaacgagacgacagaacacccacgggaccgagcttcgaatctggcaattccggttcgcttgctgtccataaaaccgcccagtctagctatcgccatgtaagcccactgcaagctacctgctttctctttgcgcttgcgttttccggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcgatccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgcccggacggccgaggtcttccgatctcctgaagccagggcagatccgtgcacagcacttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctagcttgcttggtcgttccggtaccgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcgg

SEQ ID NO: 2 - DNA, 2952 bp, Artificial
  DNA sequence of the parp-1 cDNA from Arabidopsis thaliana
atggcaagcccacataagccgtggagggcggagtatgcaaagtcgtcgaggtcttcatgtaaaacttgcaagtccgtcattaacaaggagaactttcgtcttggaaagttggttcaatctactcacttcgatggcatcatgcccatgtggaaccatgcttcttgtatactaaagaagacgaagcagataaaatcagttgatgatgttgaaggcatagaatcacttcgttgggaagatcagcaaaagattagaaaatatgtcgaatctggagcagggagtaacacaagcacaagcacaggcacaagcacgagcagtaccgctaataatgccaaactagaatatgggattgaagtgtcacaaacttctcgtgccggttgcagaaagtgtagcgaaaagatcttgaaaggagaggtacgtatattctccaagcctgaaggcccgggtaacaaaggtttgatgtggcatcacgctaaatgtttccttgaaatgtcttcctctactgaactggaaagtttgtctggatggagaagtataccagactcagaccaagaagctcttcttcccttagtgaagaaagctctgccggcagccaaaactgagacagcagaagcacgtcaaacaaattcaagagcaggcacaaaacgaaaaaatgattctgttgataacgagaagtcgaaactagcaaaaagtagttttgacatgtctacaagtggtgctttacaaccttgtagcaaagaaaaggaaatggaggcccaaactaaggaattgtgggacctgaaggatgatctgaaaaaatatgtaacatcagctgagttgcgggaaatgcttgaagtaaatgaacaaagtacaagaggatctgaacttgatctgcgtgataaatgtgctgatggcatgatgtttggcccactcgctctctgcccaatgtgctctgggcatctttctttctccggaggactttaccgatgccatggatacatctcagaatggagcaaatgttctcattccactttggatccagaccgcatcaaagggaagtggaaaatccctgacgaaacagaaaatcaattccttctgaagtggaataagtctcaaaagagtgtgaagccaaaacgtattctgcgtcctgtattgtctggcgagacatctcagggtcaaggttctaaagatgcaactgactcctcaaggagtgaaaggctagcagatcttaaagtttcaattgctggaaatactaaggaaaggcaaccatggaagaagagaattgaggaagctggtgcagagtttcatgctaatgttaaaaaaggtacaagctgtttggttgtttgtggactgacagatatcagagacgctgaaatgagaaaggcaaggaggatgaaagtggcaatcgtgagagaggattatttggttgattgttttaaaaaacagaggaaacttccatttgacaagtacaaaattgaagacactagtgagagccttgtcactgttaaagtaaaaggacgaagcgctgtgcatgaagcgtctggcctccaagagcactgtcacattcttgaagatgggaacagtatctataacacaactctgagcatgtctgatctctctaccggtatcaatagttattacatactccagataatccaagaagataaaggttcagattgttacgtatttcgtaaatggggccgagttggaaatgaaaagattggtggtaacaaagtggaggaaatgtcaaagtctgatgcggttcacgaattcaaacgtctatttcttgaaaaaaccggaaacacatgggaatcttgggaacaaaaaacgaatttccagaaacaacctggaaaatttctcccgttggacattgattatggagttaataagcaagtagccaaaaaagagccatttcagaccagtagcaaccttgctccatcattaatagaattgatgaagatgctttttgatgtggaaacttacagatctgcaatgatggagttcgagataaatatgtcagagatgccacttgggaagctcagcaaacataatatacagaagggttttgaggcattgacggagatacagaggctattgactgaaagcgacccccagcctactatgaaagaaagcttgcttgttgatgctagtaacagattttttaccatgatcccttctattcatcctcatattatccgagatgaagatgactttaagtcaaaggtgaaaatgctcgaggctctgcaggatatcgaaatagcttcaagaatagttggctttgatgttgatagcaccgaatctcttgatgataagtataagaagctgcattgcgatatctcaccacttcctcatgatagcgaagattatcgattaatcgagaagtatcttaacacaactcatgccccaacgcatacagagtggagtcttgagctagaggaagtttttgctcttgaaagagaaggagagtttgataaatatgctccccacagagaaaaacttggcaataagatgctcctatggcatggttctcgattaacgaattttgttggaatattgaaccaaggactgagaattgcacctccagaagctcctgctactggttacatgtttggaaaagggatatactttgctgaccttgtcagtaaaagtgctcagtactgctacacttgtaagaaaaatccggtgggtctaatgcttctgagtgaagttgcattgggagaaatacatgagctaacaaaagctaagtatatggataaacctccgagagggaaacactcgaccaaagggctcggcaagaaagtgcctcaagattccgagtttgccaagtggagaggtgatgtgactgttccctgtggaaaacctgtttcatcaaaggtcaaggcttctgagcttatgtacaatgagtatatcgtctacgatacagcccaggtgaagttgcagttcttgttgaaagtaaggtttaagcacaagagatga

SEQ ID NO: 3 - protein, 983 aa, Artificial
  Synthetic Construct
MASPHKPWRAEYAKSSRSSCKTCKSVINKENFRLGKLVQSTHFDGIMPMWNHASCILKKTKQIKSVDDVEGIESLRWEDQQKIRKYVESGAGSNTSTSTGTSTSSTANNAKLEYGIEVSQTSRAGCRKCSEKILKGEVRIFSKPEGPGNKGLMWHHAKCFLEMSSSTELESLSGWRSIPDSDQEALLPLVKKALPAAKTETAEARQTNSRAGTKRKNDSVDNEKSKLAKSSFDMSTSGALQPCSKEKEMEAQTKELWDLKDDLKKYVTSAELREMLEVNEQSTRGSELDLRDKCADGMMFGPLALCPMCSGHLSFSGGLYRCHGYISEWSKCSHSTLDPDRIKGKWKIPDETENQFLLKWNKSQKSVKPKRILRPVLSGETSQGQGSKDATDSSRSERLADLKVSIAGNTKERQPWKKRIEEAGAEFHANVKKGTSCLVVCGLTDIRDAEMRKARRMKVAIVREDYLVDCFKKQRKLPFDKYKIEDTSESLVTVKVKGRSAVHEASGLQEHCHILEDGNSIYNTTLSMSDLSTGINSYYILQIIQEDKGSDCYVFRKWGRVGNEKIGGNKVEEMSKSDAVHEFKRLFLEKTGNTWESWEQKTNFQKQPGKFLPLDIDYGVNKQVAKKEPFQTSSNLAPSLIELMKMLFDVETYRSAMMEFEINMSEMPLGKLSKHNIQKGFEALTEIQRLLTESDPQPTMKESLLVDASNRFFTMIPSIHPHIIRDEDDFKSKVKMLEALQDIEIASRIVGFDVDSTESLDDKYKKLHCDISPLPHDSEDYRLIEKYLNTTHAPTHTEWSLELEEVFALEREGEFDKYAPHREKLGNKMLLWHGSRLTNFVGILNQGLRIAPPEAPATGYMFGKGIYFADLVSKSAQYCYTCKKNPVGLMLLSEVALGEIHELTKAKYMDKPPRGKHSTKGLGKKVPQDSEFAKWRGDVTVPCGKPVSSKVKASELMYNEYIVYDTAQVKLQFLLKVRFKHKR